(19) United States
(12) Patent Application Publication
(10) Pub. No.: US 2014/0179973 A1
(43) Pub. Date: Jun. 26, 2014

(54) METHOD OF OLEFIN METATHESIS USING A CATALYST BASED ON A SPHERICAL MATERIAL COMPRISING OXIDISED METAL PARTICLES TRAPPED IN A MESOSTRUCTURED MATRIX

(71) Applicants: Damien Debecker, Tubize (BE); Frederic Colbeau-Justin, Ivry-Sur-Seine (FR); Clement Sanchez, Bures-Sur-Yvette (FR); Alexandra Chaumonnot, Lyon (FR); Mikael Berthod, Lyon (FR)

(72) Inventors: Damien Debecker, Tubize (BE); Frederic Colbeau-Justin, Ivry-Sur-Seine (FR); Clement Sanchez, Bures-Sur-Yvette (FR); Alexandra Chaumonnot, Lyon (FR); Mikael Berthod, Lyon (FR)

(73) Assignees: UNIVERSITE DE PARIS 6 - PIERRE ET MARIE CURIE, Paris Cedex 05 (FR); IFP ENERGIES NOUVELLES, Rueil-Malmaison Cedex (FR); UNIVERSITE CATHOLIQUE DE LOUVAIN, Louvain La Neuve (BE)

(21) Appl. No.: 14/232,313

(22) PCT Filed: Jul. 11, 2012
(86) PCT No.: PCT/FR2012/000285

§ 371 Date: Jan. 13, 2014

(30) Foreign Application Priority Data
Jul. 15, 2011 (FR) 11/02221

(51) Int. Cl. C07C 6/04

(52) U.S. Cl. CPC C07C 6/04
USPC 585/644

(57) ABSTRACT

A process for metathesis of olefins, bringing olefins into contact with a catalyst activated by heating to a temperature in the range 100° C. to 1000° C. in an atmosphere of non-reducing gas, the catalyst containing at least one inorganic material having at least two elementary spherical particles, each of which are metal oxide particles with a size of at most 300 nm and containing at least one of tungsten, molybdenum, rhenium, cobalt, tin, ruthenium, iron or titanium, alone or a mixture, the metal oxide particles being present within a mesostructured matrix of an oxide of at least one element Y: silicon, aluminium, titanium, tungsten, zirconium, gallium, germanium, tin, antimony, lead, vanadium, iron, manganese, hafnium, niobium, tantalum, yttrium, cerium, gadolinium, europium or neodymium or a mixture thereof, the matrix having pore size 1.5 to 50 nm and amorphous walls with thickness 1 to 30 nm and maximum diameter of 200 μm.

### FIELD OF THE INVENTION

The present invention relates to a process for the metathesis of olefins, which is a catalytic olefin transformation reaction consisting of exchanging the alkylidene groups of the starting olefins.

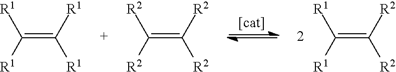

More particularly, the present invention concerns a process for the metathesis of olefins using a catalyst based on a spherical material comprising metal oxide particles trapped in a mesostructured matrix.

### PRIOR ART

The metathesis of olefins, or the reaction of redistributing alkylidene groups among themselves, is a crucial reaction in various chemical fields. In organic synthesis, this reaction, catalysed by organometallic complexes, is used to obtain various high added value molecules (drugs, etc). In petrochemicals, olefin metathesis is of great practical importance, for example for re-equilibrating light olefins obtained from steam cracking, such as ethylene, propylene or butenes. In particular, cross metathesis of ethylene with butene to provide propylene is important because of the high market demand for the latter, as described in patent applications FR 2 880 018, DE 102006039904, WO 2005/009929 and US 2002/197190.

Various types of catalysts are susceptible of being used in the metathesis reaction: either homogeneous catalysts, when their constituent elements are all soluble in the reaction medium, or heterogeneous catalysts when at least one of the elements is insoluble in said medium.

Robust and cheap heterogeneous catalysts are used for the metathesis of light olefins. A known commercial solution is the technique offered by Lummus Technology Company, based on the use of a catalyst constituted by tungsten oxide deposited on a silica support, WO₃/SiO₂, described in the publication by J. C. Mol, J. Mol. Catal. A, 2004, 213, 1, 39; R. J. Gartside; and patent CA 2733890. Said heterogeneous catalysts which are known to the skilled person are used at relatively high temperatures, generally at a temperature of more than 200° C. and are only moderately active. Metathesis catalysts based on rhenium oxide, Re₂O₇, described in the publication by M. Stoyanova et al., Appl. Catal. A, 2008, 340, 2, 242, are known to the skilled person as being more active, but they are also more expensive and less stable as they rapidly become deactivated.

One attractive solution is to use catalysts based on molybdenum oxide as described in the publication by D. P. Debecker et al., J. Catal., 2011, 277.2, 154. Molybdenum (Mo) is much cheaper than rhenium (Re) and its stability and activity are between those of rhenium (Re) and tungsten (W). Conventionally, catalysts based on molybdenum oxides (MoO₃) are prepared by impregnating an aqueous solution of a molybdenum salt such as ammonium heptamolybdate, for example, onto a support such as a silica, an alumina or a porous silica-alumina, for example, as described in the publications by D. P. Debecker et al., Catal. Today, 2011, 169, 1, 60 and J. Handzlik et al., Appl. Catal. A, 2004, 273, 1-2, 99. In this case, the difficulty consists of being sure that the molybdenum is deposited in a dispersed manner right into the interior of the pores of the support. Frequently, deposition is non-homogeneous, concentrated at the outer surface of the porous particles constituting the support and leading to the formation of inactive molybdenum oxide crystals. In the case of alumina supports, the formation of a lot of aluminium molybdate (Al₂(MoO₄)₃) following the wet impregnation step also limits the activity of the catalysts (X. Li et al., J. Mol. Catal. A, 2006, 250, 1-2, 94). One way of circumventing the formation of the inactive species mentioned above during wet impregnation is to use other molybdenum precursors. The wet impregnation step can be improved with molybdenum hydrates or with the aid of organic additives such as oxalic acid, for example, and better performances can be obtained (D. P. Debecker et al., J. Mol. Catal. A, 2011, 340, 65-76). However, impregnation remains a two-step process, necessitating prior preparation of a suitable support.

Another alternative is to carry out the synthesis using thermal spreading, i.e. heating a mechanical mixture of the support with molybdenum oxide or with bis(acetylacetonato) dioxo molybdenum, as described in the publications by D. P. Debecker et al., J. Phys. Chem. C, 2010, 114, 43, 18664 and J. Handzlik et al., Appl. Catal. A, 2006, 312, 213. By means of sublimation-deposition, molybdenum is dispersed at the surface of the support and active species are formed. That two-step process, however, produces moderately active catalysts as transfer of molybdenum to the support is not quantitative.

Aerosol spray pyrolysis, in contrast, is a one-step method which produces active catalysts composed of nanoparticles of silica-alumina adorned with molybdenum oxide (D. P. Debecker et al., J. Catal., 2011, 277.2, 154). The specific surface area of those non-porous solids remains relatively low, however, which limits the quantity of MoO₃ which can be incorporated in a dispersed manner.

The non-hydrolytic sol-gel method can also produce mixed oxides of molybdenum, silica and alumina in one step, as described in the publication by D. P. Debecker et al., Chem. Mater., 2009, 21, 13, 2817. Such catalysts have a very large specific surface area and are highly active in the metathesis of light olefins. However, the non-hydrolytic sol-gel method is complicated, requiring operations to be carried out in a controlled inert atmosphere and producing chlorinated by-products. In addition, a portion of the molybdenum is lost in the bulk of the solid and is inaccessible for the reaction at the surface.

Thus, obtaining a high performance catalyst for metathesis depends on the capacity for obtaining a material with a relatively high specific surface area and good molybdenum dispersion such that the molybdenum is accessible at the surface of the support.

Conventional sol-gel methods are usually employed for the preparation of mixed oxides, including the preparation of porous heterogeneous catalysts. The method is based on hydrolysis and condensation of alkoxides in order to generate a gel via the formation of oxo bonds. However, the reactivity of the majority of metal precursors differs greatly from that of silicon alkoxides. Even in the presence of templates such as surfactants, for example, then, properly homogeneous solids with controlled structures and textures are prevented from forming.

One way of overcoming this problem is to accelerate the kinetics of the sol-gel process by dispersing a solution of precursor and surfactant in the form of an aerosol which is rapidly dried according to the technique described in the publication by C. Boissiere et al., Adv. Mater., 2011, 23, 5, 599. Because the silica framework is formed rapidly about the template, it encapsulates the other inorganic elements, i.e. the aluminium precursors, molybdenum precursors, etc, resulting in a statistical dispersion in a mesostructured solid. This method is carried out successfully in the synthesis of various materials of interest in catalysis, as described in patent FR 2 886 636.

With the intention of improving the performances of heterogeneous catalysts for olefin metathesis, the Applicant has sought to develop novel catalysts for the olefin metathesis reaction using this latter aerosol process.

One aim of the present invention is to provide a process for the metathesis of olefins using a catalyst comprising at least one inorganic spherical material comprising metal oxide particles trapped in a mesostructured matrix, said spherical inorganic material being obtained by the particular “aerosol” synthesis technique.

One advantage of using such a catalyst for the metathesis of olefins is to allow an increased catalytic activity compared with the use of conventional heterogeneous prior art catalysts to be obtained.

### AIM OF THE INVENTION

The present invention concerns a process for the metathesis of olefins, comprising bringing said olefins into contact with a catalyst which has been activated by heating to a temperature in the range 100° C. to 1000° C. in an atmosphere of a non-reducing gas, said catalyst comprising at least one spherical inorganic material comprising metal oxide particles trapped in a mesostructured matrix, said spherical inorganic material being obtained using the particular synthesis technique known as the “aerosol” technique.

More particularly, the present invention concerns a process for the metathesis of olefins, comprising bringing said olefins into contact with a catalyst which has been activated by heating to a temperature in the range 100° C. to 1000° C. in an atmosphere of a non-reducing gas, said catalyst comprising at least one inorganic material constituted by at least two elementary spherical particles, each of said elementary spherical particles comprising metal oxide particles with a size of at most 300 nm and containing at least one metal selected from tungsten, molybdenum, rhenium, cobalt, tin, ruthenium, iron and titanium, said metal oxide particles being present within a mesostructured matrix based on an oxide of at least one element Y selected from silicon, aluminium, titanium, tungsten, zirconium, gallium, germanium, tin, antimony, lead, vanadium, iron, manganese, hafnium, niobium, tantalum, yttrium, cerium, gadolinium, europium and neodymium and a mixture of at least two of these elements, said mesostructured matrix having a pore size in the range 1.5 to 50 nm and having amorphous walls with a thickness in the range 1 to 30 nm and said elementary spherical particles having a maximum diameter of 200 μm.

Thus, the present invention concerns an olefin metathesis process using said catalyst, in particular for the following reactions, the list of which is non-limiting: ring closing by metathesis, acyclic diene polymerization by metathesis, ring opening polymerization using metathesis, acyclic olefin metathesis, cross metathesis of cyclic and acyclic olefins and the metathesis of functionalized olefins.

### DETAILED DISCLOSURE OF THE INVENTION

In accordance with the invention, the catalyst used in the olefin metathesis process comprises at least one inorganic material constituted by at least two elementary spherical particles, each of said elementary spherical particles comprising metal oxide particles with a size of at most 300 nm and containing at least one metal selected from tungsten, molybdenum, rhenium, cobalt, tin, ruthenium, iron and titanium, used alone or as a mixture, said metal oxide particles being present within a mesostructured matrix based on an oxide of at least one element Y selected from silicon, aluminium, titanium, tungsten, zirconium, gallium, germanium, tin, antimony, lead, vanadium, iron, manganese, hafnium, niobium, tantalum, yttrium, cerium, gadolinium, europium and neodymium and a mixture of at least two of these elements, said mesostructured matrix having a pore size in the range 1.5 to 50 nm and having amorphous walls with a thickness in the range 1 to 30 nm and said elementary spherical particles having a maximum diameter of 200 μm.

The element Y present in the form of an oxide in the mesostructured matrix included in each of said spherical particles of said inorganic material used in accordance with the invention is selected from silicon, aluminium, titanium, tungsten, zirconium, gallium, germanium, tin, antimony, lead, vanadium, iron, manganese, hafnium, niobium, tantalum, yttrium, cerium, gadolinium, europium and neodymium and a mixture of at least two of these elements. Preferably, said element Y present in the oxide form is selected from the group constituted by silicon, aluminium, titanium, zirconium, gallium, germanium and cerium and a mixture of at least two of these elements. Still more preferably, said element Y present in the oxide form is selected from silicon and aluminium and a mixture of these elements. In the case in which said element Y present in the oxide form is selected from silicon and aluminium and a mixture of these elements, said mesostructured matrix is preferably constituted by aluminium oxide, silicon oxide or a mixture of silicon oxide and aluminium oxide (aluminosilicate), and the associated Si/Al molar ratio is in the range 0.001 to 1000, preferably in the range 0.001 to 0.2 or in the range 3.5 to 1000; highly preferably, it is in the range 0.03 to 0.1 or in the range 8 to 25.

Said matrix based on an oxide of at least said element Y is mesostructured: it has a porosity which is organized on the mesopore scale for each of the elementary particles of the inorganic material of the invention, i.e. an organized porosity on the pore scale with a uniform diameter in the range 1.5 to 50 nm, preferably in the range 1.5 to 30 nm, and still more preferably in the range 2 to 20 nm and distributed in a homogeneous and regular manner in each of said particles (mesostructuring of the matrix). The material located between the mesopores of the mesostructured matrix is amorphous and forms walls or partitions the thickness of which is in the range 1 to 30 nm, preferably in the range 1 to 10 nm. The thickness of the walls corresponds to the distance separating a first mesopore from a second mesopore, the second mesopore being the pore which is closest to said first mesopore. The organization of the mesoporosity as described above results in a structuring of said matrix, which may be hexagonal, vermicular or cubic, preferably vermicular. It should be noted that a microporous type porosity may also be the result of imbrication of the surfactant employed during the preparation of the material used in accordance with the invention, with the inorganic wall at the organic-inorganic interface developed during mesostructuring of the inorganic component of said material of the invention. The material of the invention also has an interparticulate textural macroporosity.

In accordance with the invention, the mesostructured matrix comprised in each of said elementary spherical particles of the material used in accordance with the invention comprises metal oxide particles with a size of at most 300 nm, said metal oxide particles containing at least one metal selected from tungsten, molybdenum, rhenium, cobalt, tin, ruthenium, iron and titanium, used alone or as a mixture, and preferably containing at least one metal selected from tungsten, molybdenum and rhenium, used alone or as a mixture. Highly preferably, said metal oxide particles contain molybdenum alone.

Said metal oxide particles contain at least one metal selected from molybdenum, tungsten, rhenium, cobalt, tin, ruthenium, iron and titanium, used alone or as a mixture, said metals advantageously being in an oxygen-containing environment.

In accordance with the invention, said metal oxide particles are present within said mesostructured matrix. More precisely, said metal oxide particles are trapped in said mesostructured matrix. Said metal oxide particles are advantageously trapped in a homogeneous and uniform manner in said mesostructured matrix comprised in each of said elementary spherical particles of the material used in accordance with the invention.

In accordance with the invention, said metal oxide particles have a size of at most 300 nm, preferably at most 50 nm and still more preferably at most 3 nm. The size of said metal oxide particles is advantageously measured by transmission electron microscopy (TEM), when the size is greater than 1 nm. The absence of detection of metallic particles in TEM thus means that said metal oxide particles are less than 1 nm in size.

Said metal oxide particles advantageously represent 1% to 50% by weight, preferably 2% to 45% by weight, more preferably 2% to 40% by weight and still more preferably 2% to 25% by weight of the material used in accordance with the invention.

The inorganic material used in accordance with the invention comprises a quantity by weight of the element(s) molybdenum, tungsten, rhenium, cobalt, tin, ruthenium, iron and titanium, used alone or as a mixture, in the range 1% to 40%, expressed as the % by weight of oxide with respect to the weight of the final material in the oxide form, preferably in the range 2% to 35% by weight, more preferably in the range 2% to 30% and still more preferably in the range 2% to 20%.

Said metal oxide particles are advantageously prepared using protocols which are known to the skilled person and described below in the disclosure of the invention. Said metal oxide particles may advantageously have been pre-formed and used as they are in the process for the preparation of the inorganic material described below, or prepared, in step a) of said process for the preparation of the inorganic material, using precursors of said metal oxide particles described below in the disclosure of the invention.

Preferably, said metal oxide particles are obtained from precursors of said metal oxide particles.

The precursor(s) of said metal oxide particles is(are) preferably selected from polyoxometallates with formula (XₓMₘO_{y}Hₕ)^{q-}, and monometallic precursors. Said polyoxometallates with formula (XₓMₘO_{y}Hₕ)^{q-}, in particular isopolyanions, and heteropolyanions and monometallic precursors are described below in the present disclosure.

In a first preferred embodiment, said precursors of said metal oxide particles are selected from polyoxometallates with formula (XₓMₘO_{y}Hₕ)^{q-}, preferably from isopolyanions and heteropolyanions. In this case, said metallic precursors have a formula (XₓMₘO_{y}Hₕ)^{q-} (I) where H is the hydrogen atom, O is the oxygen atom, X is an element selected from rhenium, phosphorous, silicon, boron, nickel, tin, ruthenium, iron, titanium and cobalt and M is one or more elements selected from molybdenum, tungsten, rhenium, cobalt, tin, ruthenium, iron and titanium, x being equal to 0, 1, 2, or 4, m being equal to 5, 6, 7, 8, 9, 10, 11, 12 and 18, y being in the range 17 to 72, h being in the range 0 to 12 and q being in the range 1 to 20, y, h and q being whole numbers.

In the definition of this formula, in the context of the present invention, the intention is that the elements H, X, M and O are present in the structure of the polyoxometallates.

Preferably, said precursors of said metal oxide particles in the form of polyoxometallates with formula (I) are preferably selected from isopolyanions and heteropolyanions (denoted HPA). Preferably, said precursors with formula (I) are heteropolyanions.

Said precursors with formula (I), preferably in the form of heteropolyanions, are salts carrying a negative charge q which is compensated by positively charged counter-ions of an identical or different nature. The counter-ions are advantageously provided by metallic cations, in particular cations of metals from group VIII such as Co²⁺, Ni²⁺, protons H⁺ and/or ammonium cations NH₄⁺. When all of the counter-ions are protons H⁺, the term “heteropolyacid” is generally used to designate the form in which said precursors with formula (I) are present. An example of such a heteropolyacid is phosphomolybdic acid (3H⁺, PMo₁₂O₄₀³⁻), or indeed phosphotungstic acid (3H⁺, PW₁₂O₄₀³⁻).

The isopolyanions and heteropolyanions used as metallic precursors of said metal oxide particles are comprehensively described in the publication “Heteropoly and Isopoly Oxometalates”, Pope, Ed Springer-Verlag, 1983.

When isopolyanions are used, the element X occurring in general formula (I) above is absent and x=0. The element M is one or more elements advantageously selected from molybdenum, tungsten, rhenium, cobalt, ruthenium, tin, iron, titanium (Heteropoly and Isopoly Oxometalates, Pope, Ed Springer-Verlag, 1983, chapter 4, Table 4.1). Preferably, the element M is selected from molybdenum and tungsten, used alone or as a mixture. Molybdenum and/or tungsten as the element M present in said general formula (I) is/are advantageously mixed with one or more elements M selected from cobalt, rhenium, titanium, ruthenium, tin and iron (partial substitution of one or more elements M=Mo and W with Re, Ti, Ru, Fe, Sn and/or Co). Preferably, the m atoms of M present in general formula (I) are all exclusively either atoms of Mo, or atoms of W, or a mixture of Mo and W atoms, or a mixture of Mo and Co atoms, or a mixture of Mo and Re atoms, or a mixture of W and Re atoms, or a mixture of Mo and W atoms. The index m is equal to 5, 6, 7, 8, 9, 10, 11, 12 and 18. Still more preferably, m is equal to 6, 7 and 12. In the particular case in which the element M is molybdenum (Mo), the value of m is preferably 7. In another particular case where the element M is tungsten (W), the value of m is preferably 12. In general formula (I), O designates the element oxygen with 17≦y≦48, q designates the charge of the isopolyanion, with 3≦q≦12 and H is the element hydrogen with h=0 to 12. A preferred isopolyanion has the formula H₂W₁₂O₄₀⁶⁻ (h=2, m=12, y=40, q=6) or the formula Mo₇O₂₄⁶⁻ (h=0, m=7, y=24, q=6).

When using heteropolyanions (denoted HPA), the element X is the central atom in the heteropolyanion structure and is selected from rhenium, phosphorous, silicon, boron, nickel, tin, ruthenium, iron, titanium and cobalt with x=1 or 2. The element M is a metal atom which advantageously occurs systematically in octahedral coordination in the heteropolyanion structure. The element M is one or more elements advantageously selected from molybdenum, tungsten, cobalt, rhenium, tin, ruthenium, iron and titanium. More preferably, the element M is one or more elements selected from molybdenum and tungsten. The Molybdenum and/or tungsten acting as the element M present in said general formula (I) is/are advantageously mixed with one or more elements M selected from cobalt, rhenium, titanium, tin, ruthenium and iron (partial substitution of one or more elements M=Mo and W with Re, Ti, Ru, Fe, Sn and/or Co). Preferably, the m atoms of M present in general formula (I) are all exclusively either atoms of Mo, or atoms of W, or a mixture of atoms of Mo and W, or a mixture of atoms of Mo and Co, or a mixture of atoms of Mo and Re, or a mixture of atoms of W and Re. The index m is equal to 5, 6, 7, 8, 9, 10, 11, 12 and 18 and preferably equal to 5, 6, 9, 10, 11, 12, 18. In the general formula (I), O designates the element oxygen with y in the range 17 to 72, preferably in the range 23 to 42, q designates the charge of the heteropolyanion with 1≦q≦20, preferably 3≦q≦12, and H is the element hydrogen with h=0 to 12.

A first preferred category of heteropolyanions as metallic precursors for said metal oxide particles is such that said heteropolyanions have the formula XM₆O₂₄Hₕ^{q-} (with x=1, m=6, y=24, q=3 to 12 and h=0 to 12) and/or the formula X₂M₁₀O₃₈Hₕ^{q-} (with x=2, m=10, y=38, q=3 to 12 and h=0 to 12) with H, X, M, O, h, x, m, y and q having the same definitions as those given in the general formula (I) above. Such heteropolyanions are known as Anderson heteropolyanions (Nature, 1937, 150, 850). They comprise 7 octahedra located in the same plane and connected together via the edges: 6 octahedra surround the central octahedron containing the heteroelement X. The heteropolyanion CoMo₆O₂₄H₆³⁻ is a good example of an Anderson heteropolyanion, the Co being the heteroelement X of the HPA structure. In the case in which the HPA contains cobalt (X=Co) and molybdenum (M=Mo) within its structure, it is preferably dimeric. A mixture of two monomeric and dimeric forms of said HPA may also be used. Highly preferably, the Anderson HPA used to obtain the material used in accordance with the invention is a dimeric HPA comprising cobalt and molybdenum within its structure and the counter ion for the HPA salt may be cobalt, Co^{II}₃[Co^{III}₂Mo₁₀O₃₈H₄].

A second preferred category of heteropolyanion as metallic precursors for said metal oxide particles is such that said heteropolyanions have the formula XM₁₂O₄₀Hₕ^{q-} (x=1, m=12, y=40, h=0 to 12, q=3 to 12) and/or the formula XM₁₁O₃₉Hₕ^{q-} (x=1, m=11, y=39, h=0 to 12, q=3 to 12) with H, X, M, O, h, x, m, y and q having the same definitions as those given in the general formula (I) above. Heteropolyanions with formula XM₁₂O₄₀Hₕ^{q-} are heteropolyanions with a Keggin structure and heteropolyanions with formula XM₁₁O₃₉Hₕ^{q-}are heteropolyanions with a lacunary Keggin structure. The heteropolyanions with a Keggin structure are obtained, for variable pH ranges, using the production pathways described in the publication by A. Griboval, P. Blanchard, E. Payen, M. Fournier, J. L. Dubois, Chem. Lett., 1997, 12, 1259. Keggin structure heteropolyanions are also known in substituted forms in which a metallic element, preferably cobalt or rhenium, substitutes for the metal M present in the formula XM₁₂O₄₀Hₕ^{q-}: examples of such substituted Keggin species are the heteropolyanions PCoMo₁₁O₄₀H⁶⁻ (one Mo atom substituted with a Co atom) and PW₁₁{ReN}]O₃₉⁴⁻ (C. Dablemont et al., Chemistry, 2006, 12, 36, 9150). The PCoMo₁₁O₄₀H⁶⁻ species is prepared, for example, using the protocol described in the publication by L. G. A. van de Water et al. J. Phys. Chem. B, 2005, 109, 14513. Other substituted Keggin heteropolyanion species are the species PMo₃W₉O₄₀³⁻, PMo₆W₆O₄₀³⁻, PMo₉W₃O₄₀³⁻.

A preferred precursor for said metal oxide particles is the Keggin heteropolyanion with formula H₃PMo₁₂O₄₀.

A third preferred category of heteropolyanions as metallic precursors of said metal oxide particles is such that said heteropolyanions have the formula P₂Mo₅O₂₃Hₕ^{(6-h)-}, with h=0, 1 or 2. Such heteropolyanions are known as Strandberg heteropolyanions. The preparation of Strandberg HPAs is described in the article by W-C. Cheng et al. J. Catal., 1988, 109, 163.

Advantageously, the metallic precursors of said metal oxide particles used to obtain the material used in accordance with the invention are in the form of heteropolyanions selected from the first, the second and/or the third category described above. In particular, said precursors may constitute a mixture of HPAs with different formulae belonging to the same category, or a mixture of heteropolyanions belonging to different categories.

The isopolyanions or heteropolyanions as described above in the present disclosure are prepared using syntheses which are known to the skilled person or are commercially available.

In general and in a manner known to the skilled person, the isopolyanions are formed by reacting together oxoanions of the MO₄ⁿ⁻ type (the value of n depends on the nature of M: n is preferably equal to 2 when M=Mo or W) where M is one or more elements selected from molybdenum, tungsten, cobalt, rhenium, tin, ruthenium, iron and titanium. As an example, molybdenum compounds are well known for this type of reaction since, depending on the pH, the molybdenum compound in solution may be in the MoO₄²⁻ form or in the form of an isopolyanion MO₇O₂₄⁶⁻ obtained in accordance with the reaction: 7 MoO₄²⁻+8H⁺→Mo₇O₂₄⁶⁻+4 H₂O. Regarding tungsten-based compounds, potential acidification of the reaction medium may result in generating the 12-fold condensed α-metatungstate: 12 WO₄²⁻+18H⁺→H₂W₁₂O₄₀⁶⁻+8 H₂O. These isopolyanion species, in particular the species Mo₇O₂₄⁶⁻ and H₂W₁₂O₄₀⁶⁻, are advantageously employed as precursors of the metal oxide particles in the process used in the invention. The preparation of isopolyanions has been amply described in the publication “Heteropoly and Isopoly Oxometallates”, Pope, Ed Springer-Verlag, 1983 (Chapter II, pages 15 and 16).

In general and in a manner known to the skilled person, heteropolyanions are obtained by polycondensation of MO₄ⁿ⁻ type oxoanions (the value of n depending on the nature of M: n is preferably equal to 2 when M=Mo or W) around one (or more) oxoanion(s) of the type XO₄^{q-}, (the value of q depending on the nature of M, the charge q being dictated by the octet rule and the nature of X), M being one or more elements selected from molybdenum, tungsten, rhenium, cobalt, tin, ruthenium, iron and titanium and X being an element selected from rhenium, phosphorous, silicon, boron, nickel, tin, ruthenium, iron, titanium and cobalt. Next, water molecules are eliminated and oxo bridges are created between the atoms X and M. These condensation reactions are governed by various experimental factors such as pH, the concentration of the species in solution, the nature of the solvent and the ratio of the number of atoms M/X. The preparation of heteropolyanions has been amply described in the publication “Heteropoly and Isopoly Oxometallates”, Pope, Ed Springer-Verlag, 1983 (chapter II, pages 15 and 16).

In a second preferred embodiment, said precursors of said metal oxide particles are selected from monometallic precursors. Preferably, said monometallic precursors are based on molybdenum, tungsten, rhenium, cobalt, tin, ruthenium, iron and titanium, and preferably based on tungsten, molybdenum and rhenium; highly preferably, said monometallic precursors are based on molybdenum. It is also possible to use at least two monometallic precursors, each of said precursors being based on a different metal selected from molybdenum, tungsten, rhenium, cobalt, tin, ruthenium, iron and titanium.

In the preferred case in which said monometallic precursors comprise tungsten, molybdenum, rhenium, cobalt, tin, ruthenium, iron and/or titanium, at least one monometallic precursor formed from one of the following species is advantageously used in the process of the invention: alcoholate or phenolate species (W—O bond, Mo—O bond), amide type species (W—NR₂ bond, Mo—NR₂ bond), halide type species (W—Cl bond, Mo—Cl bond for example), imido type species (W═N—R bond, Mo═N—R bond), oxo type species (W═O bond, Mo=O bond), or hydride type species (W—H bond, Mo—H bond). Advantageously, said monometallic precursor is selected from the following species: (NH₄)₂MO₄ (M=Mo, W), Na₂MO₄ (M=Mo, W), H₂MoO₄, (NH₄)₂MS₄ (M=Mo, W), MoO₂Cl₂, MoCl₄, MoCl₅, Na₂MoO₄, (NH₄)Mo₂O₇, Mo(NO₂)Cl₂, W(OEt)₅, W(Et)₆, WCl₆, WCl₄, WCl₂, WPhCl₃, NH₄ReO₄, Re₂(CO)₁₀, HReO₄, ReCl₅, Bu₄Sn, SnCl₄, Sn(C₂H₅)₄, RuCl₃, Ru₃(CO)₁₂, Ru(NO)(NO₃)₃, 2CoCO₃.3Co(OH)₂.H₂O, Co(NO₃)₂.6H₂O, CoCl₂, Fe(NO₃)₃, FeSO₄, FeCl₂.4H₂O, FeCl₂, FeCl₃, Fe(CO)₅, Fe₂O₃, Fe₃O₄, TiCl₄, Ti(OCOCCl₃)₃, TiF₄, Ti(OⁱPr)₄, TiCl(OⁱPr)₃. However, any monometallic precursor which is familiar to the skilled person may be employed.

In accordance with the invention, said elementary spherical particles constituting the material used in accordance with the invention have a maximum diameter equal to 200 μm, preferably less than 100 μm, advantageously in the range 30 nm to 50 μm, highly advantageously in the range 50 nm to 30 μm and still more advantageously in the range 50 nm to 10 μm. More precisely, they are present in the material used in accordance with the invention in the form of powder, beads, pellets, granules, extrudates (cylinders which may or may not be hollow, multilobed cylinders, for example with 2, 3, 4 or 5 lobes, or twisted cylinders) or rings. The inorganic material contained in the catalyst used in the process of the invention advantageously has a specific surface area in the range 50 to 1100 m²/g, highly advantageously in the range 50 to 600 m²/g and still more advantageously in the range 50 to 500 m²/g.

### Process for Preparing the Inorganic Material Contained in the Catalyst Used in the Process of the Invention

The inorganic material contained in the catalyst used in the process of the invention is advantageously prepared using the preparation process described below.

Said process comprises at least the following steps:

a) mixing in solution:
at least one surfactant;
at least one precursor of at least one element Y selected from silicon, aluminium, titanium, tungsten, zirconium, gallium, germanium, tin, antimony, lead, vanadium, iron, manganese, hafnium, niobium, tantalum, yttrium, cerium, gadolinium, europium and neodymium and a mixture of at least two of these elements;
said metal oxide particles and/or at least one precursor of said metal oxide particles containing at least one metal selected from molybdenum, tungsten, rhenium, cobalt, tin, ruthenium, iron and titanium, used alone or as a mixture;
   b) aerosol atomisation of said solution obtained in step a) in order to result in the formation of spherical liquid droplets;
   c) drying said droplets;
   d) eliminating at least said surfactant in order to obtain said mesostructured inorganic material in which said metal oxide particles are trapped.

In the case of using at least one precursor of said metal oxide particles in step a), said step d) for eliminating at least said surfactant, preferably by heat treatment, is used to transform at least said metallic precursor into said metal oxide particles which constitute the inorganic material of the catalyst used in the process of the invention.

At the end of said preparation process, an inorganic material is obtained which is then optionally shaped for use as the catalyst in the process of the invention, said inorganic material comprising metal oxide particles trapped in each of the mesostructured matrices present in each of the elementary spherical particles constituting said inorganic material.

The surfactant used to prepare the mixture in accordance with step a) of said preparation process is an ionic or non-ionic surfactant or a mixture of the two. Preferably, the ionic surfactant is selected from phosphonium and ammonium ions, and highly preferably from quaternary ammonium salts such as cetyltrimethylammonium bromide (CTAB). Preferably, the non-ionic surfactant may be any copolymer having at least two portions with different polarities, endowing them with amphiphilic macromolecular properties. These copolymers may comprise at least one block forming part of the following non-exhaustive list of polymer families: fluorinated polymers (—[CH₂—CH₂—CH₂—CH₂—O—CO—R1-, in which R1=C₄F₉, C₈F₁₇, etc.), biological polymers such as polyamino acids (polylysine, alginates, etc.), dendrimers, polymers constituted by chains of poly(alkylene oxide). In general, any copolymer with an amphiphilic nature which is known to the skilled person may be used (S. Förster, M. Antionnetti, Adv. Mater, 1998, 10, 195-217; S. Förster, T. Plantenberg, Angew. Chem. Int. Ed, 2002, 41, 688-714; H. Cölfen, Macromol. Rapid Commun, 2001, 22, 219). Preferably, in the context of the present invention, a block copolymer is used which is constituted by chains of poly(alkylene oxide). Said block copolymer is preferably a block copolymer containing two, three or four blocks, each block being constituted by one chain of poly(alkylene oxide). For a two-block copolymer, one of the blocks is constituted by a chain of poly(alkylene oxide) with a hydrophilic nature and the other block is constituted by a poly(alkylene oxide) chain with a hydrophobic nature. For a three-block copolymer, at least one of the blocks is constituted by a poly(alkylene oxide) chain with a hydrophilic nature, while at least one of the other blocks is constituted by a poly(alkylene oxide) chain with a hydrophobic nature. Preferably, in the case of a three-block copolymer, the poly(alkylene oxide) chains with a hydrophilic nature are chains of poly(ethylene oxide) denoted (PEO)_{w} and (PEO)_{z} and the chains of poly(alkylene oxide) with a hydrophobic nature are chains of poly(propylene oxide) denoted (PPO)_{y}, chains of poly(butylene oxide), or mixed chains wherein each chain is a mixture of several alkylene oxide monomers. Highly preferably, in the case of a three-block copolymer, a compound with formula (PEO)_{w}-(PPO)_{y}-(PEO)_{z} is used, where w is in the range 5 to 300, y is in the range 33 to 300 and z is in the range 5 to 300. Preferably, the values for w and z are identical. Highly advantageously, a compound in which w=20, y=70 and z=20 (P123) is used and a compound in which w=106, y=70 and z=106 (F127) is used. Commercially available non-ionic surfactants with the names Pluronic (BASF), Tetronic (BASF), Triton (Sigma), Tergitol (Union Carbide), Brij (Aldrich) may be used as non-ionic surfactants. For a four-block copolymer, two of the blocks are constituted by a chain of poly(alkylene oxide) with a hydrophilic nature and the other two blocks are constituted by a chain of poly(alkylene oxide) with a hydrophobic nature. Preferably, to prepare the mixture in accordance with step a) of the process for the preparation of the material of the invention, a mixture of an ionic surfactant such as CTAB and a non-ionic surfactant such as P123 or F127 is used.

In accordance with step a) of said preparation process, the precursor(s) of at least one element Y selected from the group constituted by silicon, aluminium, titanium, tungsten, zirconium, gallium, germanium, tin, antimony, lead, vanadium, iron, manganese, hafnium, niobium, tantalum, yttrium, cerium, gadolinium, europium and neodymium, and a mixture of at least two of these elements, preferably selected from the group constituted by silicon, aluminium, titanium, zirconium, gallium, germanium and cerium and a mixture of at least two of these elements is(are) inorganic oxide precursor(s) which are well known to the skilled person. The precursor(s) of at least said element Y may be any compound comprising the element Y and capable of liberating that element in solution, for example in aquo-organic solution, preferably in aquo-organic acid solution, in the reactive form. In the preferred case in which Y is selected from the group constituted by silicon, aluminium, titanium, zirconium, gallium, germanium and cerium and a mixture of at least two of these elements, the precursor(s) of at least said element Y is(are) advantageously an inorganic salt of said element Y with formula YZₙ, (n=3 or 4), Z being a halogen, the group NO₃ or a perchlorate; preferably, Z is chlorine. The precursor(s) of at least said element Y under consideration may also be (an) alkoxide precursor(s) with formula Y(OR)ₙ where R=ethyl, isopropyl, n-butyl, s-butyl, t-butyl, etc. or a chelated precursor such as Y(C₅H₈O₂)ₙ, with n=3 or 4. The precursor(s) of at least said element Y under consideration may also be (an) oxide(s) or (a) hydroxide(s) of said element Y. Depending on the nature of the element Y, the precursor of the element Y under consideration may also be in the form YOZ₂, Z being a monovalent anion such as a halogen, or the group NO₃. Preferably, said element(s) Y is(are) selected from the group constituted by silicon, aluminium, titanium, zirconium, gallium, germanium and cerium and a mixture of at least two of these elements. Highly preferably, the mesostructured oxide matrix comprises, and preferably is constituted by, aluminium oxide, silicon oxide (Y=Si or Al) or comprises, preferably is constituted by, a mixture of silicon oxide and aluminium oxide (Y=Si+Al). In the particular case in which Y is silicon or aluminium or a mixture of silicon and aluminium, the silica and/or alumina precursors used in step a) of the process for the preparation of the material in accordance with the invention are inorganic oxide precursors which are well known to the skilled person. The silica precursor is obtained from any source of silica, advantageously from a sodium silicate precursor with formula Na₂SiO₃, a chlorinated precursor with formula SiCl₄, an alkoxide precursor with formula Si(OR)₄ where R=H, methyl or ethyl, or a chloralkoxide precursor with formula Si(OR)₄₋ₐClₐ where R=H, methyl or ethyl, a being in the range between 0 and 4. The silica precursor may also advantageously be an alkoxide precursor with formula Si(OR)₄₋ₐR′ₐ where R=H, methyl or ethyl and R′ is an alkyl chain or a functionalized alkyl chain, for example functionalized by a thiol, amino, β-diketone or sulphonic acid group, a being in the range between 0 and 4. A preferred silica precursor is tetraethylorthosilicate (TEOS). The alumina precursor is advantageously an inorganic salt of aluminium with formula AlZ₃, Z being a halogen or the group NO₃. Preferably, Z is chlorine. The alumina precursor may also be an alkoxide precursor with formula Al(OR″)₃ where R″=ethyl, isopropyl, n-butyl, s-butyl or t-butyl, or a chelated precursor such as aluminium acetylacetonate (Al(CH₇O₂)₃). The alumina precursor may also be an oxide or an aluminium hydroxide.

Said surfactant and at least said precursor of at least one element Y used in the mixture for said step a) may advantageously, independently of each other, be dissolved prior to step a), said solutions then being mixed in said step a).

In accordance with step a) of said preparation process, either metal oxide particles or at least one precursor of said metal oxide particles is/are introduced into said mixture for step a).

In the case in which at least one precursor of said metal oxide particles is introduced into said mixture for step a), said mixture in solution for step a) may advantageously be produced in accordance with a first implementation described below.

In a first implementation of step a) of said preparation process, prior to said step a), precursor(s) of said metal oxide particles are dissolved or formed in solution. In the case in which the precursor(s) of said metal oxide particles are dissolved or formed in solution, prior to step a), said solution is then introduced into the mixture for said step a). Preferably, the solvent used for dissolving the precursor or precursors or for forming it/them in solution is aqueous and the solution obtained prior to step a) once the precursor(s) has/have been dissolved or formed in solution containing said precursors is clear and has a neutral, basic or acidic pH, preferably acidic.

In order to carry out said first implementation of said preparation process in the case in which said metal oxide particle precursors are polyoxometallates with formula (I) and preferably heteropolyanions, said precursors are either prepared from the precursors necessary for their production, which prior to carrying out said step a) are dissolved in a solvent, preferably aqueous, in order to form a clear solution with a neutral or acidic, preferably acidic pH before being introduced into said mixture of step a), or are dissolved in a solvent, preferably aqueous, before being introduced into said mixture of said step a).

In the case in which the precursors of the metal oxide particles are polyoxometallates with formula (I), preferably heteropolyanions, and are prepared from the precursors necessary for their production, which are dissolved prior to carrying out said step a), it is advantageous to add at least one complexing agent to the solution in order to facilitate obtaining an atomisable mixture during step a) with a view to carrying out said step b) of said preparation process. Said complexing agent may advantageously be any compound which is known to the skilled person for its possible complexing with precursors of the heteropolyanion type. As an example, said complexing agent is urea, thiourea or acetyl acetonate.

In the case in which metal oxide particles are introduced into said mixture for step a), said mixture in solution for step a) may advantageously be produced in accordance with a second implementation as described below.

In this second implementation of step a) of said preparation process, a colloidal solution of said metal oxide particles is prepared prior to step a) employing operating protocols which are well known to the skilled person, said colloidal solution in which the metal oxide particles are dispersed then being introduced into the mixture for said step a). The metal oxide particles dispersed in a colloidal solution may be obtained using a first method consisting of a step for hydroxylation of a metal cation obtained from the salt of said metal M, M advantageously being selected from molybdenum, tungsten, rhenium, cobalt, tin, ruthenium, iron and titanium, by acid-base reaction (addition of an acid or a base) or by a thermohydrolysis reaction followed by a condensation step which involves olation or oxolation reactions producing said metal oxide particles.

It is also possible to obtain these same metal oxide particles dispersed in colloidal solution using a second method carried out starting from hydrolysis and condensation reactions of alkoxide precursors of said metal with formula M(OR)ₙ, where M is advantageously selected from molybdenum, tungsten, rhenium, cobalt, tin, ruthenium, iron and titanium and R is an alkyl group, generally controlled by the presence in solution of a complexing agent (sol-gel pathway). In wishing to carry out one or other of these methods, the skilled person would be bound to refer to the following works and publications: J-P. Jolivet, Metal Oxide Chemistry and Synthesis. From Solution to Solid State, J. Wiley and Sons, Chichester, 2000; F. Schüth, K. S. W. Sing, J. Weitkamp, Handbook of Porous Solids, Wiley-VCH, 2002, Volume 3; J. Livage, C. Sanchez, J. Non Crystalline Solids, 1992, 145, 11.

Finally, a third method for obtaining metal oxide particles dispersed in colloidal solution consists of carrying out non-hydrolytic processes generally at low temperature, the systems studied being constituted by a precursor such as a salt or an alkoxide, for example, in an organic solvent such as benzyl alcohol, for example, (M. Niederberger, M. H. Bard, G. D. Stucky, J. Am. Chem. Soc., 2002, 124, 46, 13642; P. H. Mutin and A. Vioux, Chem. Mater., 2009, 21, 4, 582).

In the case in which metal oxide particles or at least one precursor of said metal oxide particles are introduced into said mixture for step a), said mixture in solution for step a) may advantageously be produced in accordance with a third implementation described below. In a third implementation of step a) of said preparation process, said metal oxide particles and/or at least one precursor of said metal oxide particles or a precursor of a precursor of the polyoxometallate type with formula (I) containing at least one metal selected from molybdenum, tungsten, rhenium, cobalt, tin, ruthenium, iron and titanium, used alone or as a mixture, are introduced in the isolated form into the solution of the mixture for step a) of said preparation process.

Said metal oxide particles and at least one precursor of said metal oxide particles used in step a) of said preparation process may also be commercially available.

The solution in which at least one surfactant, at least one precursor of at least one element Y, said metal oxide particles and/or at least one precursor of said metal oxide particles in accordance with step a) of said process for the preparation of a catalyst used in the process of the invention may advantageously be acidic, neutral or basic. Preferably, said solution is acidic and has a maximum pH of 3, preferably in the range 0 to 2. Non-exhaustive examples of acids which may be used to obtain an acidic solution are hydrochloric acid, sulphuric acid and nitric acid. Said solution in accordance with said step a) may be aqueous, or it may be a water-organic solvent mixture, the organic solvent preferably being a polar solvent which is miscible with water, in particular an alcohol, preferably ethanol. Said solution in accordance with said step a) of the preparation process of the invention may also be practically organic, preferably practically alcoholic, the quantity of water being such that hydrolysis of the inorganic precursors is ensured (stoichiometric quantity). Highly preferably, said solution in said step a) of the preparation process used in accordance with the invention in which at least one surfactant, at least one precursor of at least one element Y, said metal oxide particles and/or at least one precursor of said metal oxide particles are mixed is an acidic aquo-organic mixture, highly preferably a water-alcohol acidic mixture.

The initial concentration of surfactant introduced into the mixture in accordance with said step a) of said process for the preparation of a catalyst used in the process of the invention is defined by c₀, and c₀ is defined with respect to the critical micellar concentration (c_{mc}) which is familiar to the skilled person. The c_{mc} is the limiting concentration beyond which self-assembly of molecules of surfactant occurs in the solution. The concentration c₀ may be lower than, equal to or higher than the c_{mc}; preferably, it is lower than the c_{mc}. In a preferred implementation of the process for the preparation of the material used in accordance with the invention, the concentration c₀ is less than c_{mc} and said solution envisaged in step a) of said process used in accordance with the invention is an acidic water-alcohol mixture. In the case in which the solution envisaged in step a) of the preparation process used in accordance with the invention is a water-organic solvent mixture, preferably acidic, during said step a) of the preparation process used in accordance with the invention, the concentration of surfactant at the origin of the mesostructuring of the matrix should preferably be lower than the critical micellar concentration so that evaporation of said aquo-organic solution, preferably acidic, during step b) of the preparation process used in accordance with the invention by the aerosol technique causes a phenomenon of micellization or self-assembly, resulting in mesostructuring of the matrix of the material used in accordance with the invention around said metal oxide particles and/or at least one precursor of said metal oxide particles. When c₀<c_{mc}, the mesostructuring of the matrix of the material prepared using the process described above is consecutive to a gradual concentration, in each droplet, of at least the precursor of said element Y and the surfactant, up to a concentration of surfactant of c>c_{mc} resulting from evaporation of the aquo-organic solution, preferably acidic.

In general, the increase in the combined concentration of at least one precursor of said hydrolysed element Y and the surfactant causes precipitation of at least said hydrolysed precursor of said element Y around the self-organized surfactant and as a consequence, structuring of the matrix of the material used in accordance with the invention. The interactions of the inorganic species among themselves, organic/inorganic phases and the organic species among themselves result, via a cooperative self-assembly mechanism, in condensation of at least said precursor of said hydrolysed element Y about the self-organized surfactant. During this self-assembly phenomenon, said metal oxide particles and/or at least one precursor of said metal oxide particles become trapped in said mesostructured matrix based on an oxide of at least said element Y included in each of the elementary spherical particles constituting the material used in accordance with the invention.

The aerosol technique is particularly advantageous for carrying out said step b) of said process for the preparation of a catalyst used in the process of the invention so as to constrain the reagents present in the initial solution to interact together; loss of material apart from solvents, i.e. the solution, preferably the aqueous solution, preferably acidic, and optionally supplemented with a polar solvent, is not possible, and so the totality of said element(s) Y, said metal oxide particles and/or at least one precursor of said metal oxide particles initially present is completely conserved throughout the preparation process used in accordance with the invention, instead of potentially being eliminated during filtration and washing steps encountered in conventional synthesis processes which are known to the skilled person.

The step for atomization of the solution in accordance with said step b) of said process for the preparation of the catalyst used in the process of the invention produces spherical droplets. The size distribution of these droplets is of the log normal type. The aerosol generator used in the context of the present invention is a commercial model 9306A apparatus provided by TSI which has a 6-jet atomizer. Atomization of the solution is carried out in a chamber into which a vector gas, preferably a O₂/N₂ mixture (dry air), is fed at a pressure P equal to 1.5 bars. The diameter of the droplets varies as a function of the aerosol apparatus employed. In general, the diameter of the droplets is in the range 90 nm to 600 μm.

In accordance with step c) of said preparation process, said droplets are then dried. Drying is carried out by transporting said droplets via the vector gas, preferably the O₂/N₂ mixture, in PVC tubes, which results in gradual evaporation of the solution, for example the acidic aquo-organic solution obtained during said step a), and thus to the production of elementary spherical particles. Said drying is completed by passing said particles into an oven the temperature of which can be adjusted, the normal temperature range being from 50° C. to 600° C., preferably 80° C. to 500° C., the residence time for these particles in the oven being of the order of one second. The particles are then collected on a filter. A pump placed at the end of the circuit encourages the species to be channeled into the experimental aerosol device. Drying the droplets in step c) of said preparation process is advantageously followed by passage through the oven at a temperature in the range 50° C. to 150° C.

In accordance with step d) of said preparation process, elimination of the surfactant introduced in said step a) of said preparation process is advantageously carried out by heat treatment, preferably by calcining in air (optionally enriched in O₂) in a temperature range of 300° C. to 1000° C., preferably in a range of 500° C. to 600° C. for a period of 1 to 24 hours, preferably for a period of 3 to 15 hours. Said step d) can also be used to transform at least one precursor into said metal oxide particles constituting the inorganic material of the catalyst used in the process of the invention.

The mesostructured inorganic material used in the process of the invention as obtained and constituted by elementary spherical particles comprising metal oxide particles trapped in a mesostructured matrix based on an oxide of at least one element Y may be shaped into the form of a pelletized, crushed, sieved powder, beads, pellets, granules, extrudates (cylinders which may or may not be hollow, multilobed cylinders with 2, 3, 4 or 5 lobes for example, twisted cylinders), or rings, etc., these shaping operations being carried out using conventional techniques which are known to the skilled person. Preferably, said mesostructured inorganic material is obtained in the form of a powder, which is constituted by elementary spherical particles with a maximum diameter of 200 μm. In the case in which said mesostructured inorganic material is obtained in the powder form, it may be used directly as a catalyst in the process of the invention without any prior shaping step.

The operation for shaping said inorganic material consists of mixing said mesostructured material with at least one porous oxide material which acts as a binder. Said porous oxide material is preferably a porous oxide material selected from the group formed by alumina, silica, silica-alumina, magnesia, clays, titanium oxide, zirconium oxide, lanthanum oxide, cerium oxide, aluminium phosphates, boron phosphates and a mixture of at least two of the oxides cited above. Said porous oxide material may also be selected from alumina-boron oxide, alumina-titanium oxide, alumina-zirconia and titanium oxide-zirconia mixtures. The aluminates, for example magnesium, calcium, barium, manganese, iron, cobalt, nickel, copper or zinc aluminates, as well as mixed aluminates, for example those containing at least two of the metals cited above, are advantageously used as the porous oxide material. It is also possible to use titanates, for example zinc, nickel or cobalt titanates. It is also advantageously possible to use mixtures of alumina and silica and mixtures of alumina with other compounds such as elements from group VIB, phosphorus, fluorine or boron. It is also possible to use simple, synthetic or natural clays of the dioctahedral 2:1 phyllosilicate or trioctahedral 3:1 phyllosilicate type such as kaolinite, antigorite, chrysotile, montmorillonite, beidellite, vermiculite, talc, hectorite, saponite or laponite. These clays may optionally be delaminated. Advantageously, it is also possible to use mixtures of alumina and clay and mixtures of silica-alumina and clay. Various mixtures using at least two of the compounds cited above are also suitable for carrying out the role of a binder.

At the end of the shaping step, the catalyst is obtained which comprises at least one spherical inorganic material comprising metal oxide particles trapped in a mesostructured matrix used in accordance with the invention.

### Characterization Techniques

The material used in accordance with the invention is characterized by a number of analytical techniques, in particular by small angle X-ray diffraction (small angle XRD), wide angle X-ray diffraction (XRD), nitrogen volumetric analysis (BET), transmission electron microscopy (TEM) possibly coupled with X ray analysis, scanning electron microscopy (SEM), X-ray fluorescence (XRF), nuclear magnetic resonance (NMR), and small angle X ray diffusion (SAXS). The presence of metal oxide particles as described further on in the present disclosure is demonstrated by various techniques, in particular by Raman, UV-visible or infrared spectroscopy, as well as by microanalysis. Techniques such as nuclear magnetic resonance (NMR) or paramagnetic electron resonance (PER) may also be used, depending on the precursors employed.

The techniques described to characterize the metal oxide particles can also be used to characterize the precursors of said metal oxide particles.

The small angle X-ray diffraction technique (values for the angle 2θ in the range 0.5° to 5°) can be used to characterize periodicity on a nanometric scale generated by the organized mesoporosity of the mesostructured matrix of the material used in accordance with the invention. In the following disclosure, the X-ray analysis is carried out on a powder with a diffractometer operating in reflection mode and provided with a back monochromator using the copper radiation line (wavelength 1.5406 Å). The peaks normally observed on the diffractograms corresponding to a given value of the angle 2θ are associated with the lattice spacings d₍ₕₖₗ₎ which are characteristic of the structural symmetry of the material ((hkl) being the Miller indices of the reciprocal lattice) by Bragg's law: 2 d₍ₕₖₗ₎*sin (θ)=n*λ. This indexation then allows the lattice parameters (abc) of the direct lattice to be determined, the value of these parameters being a function of the hexagonal, cubic or vermicular structure obtained.

The wide angle X-ray diffraction technique (values for the angle 2θ in the range 6° to 100°) can be used to characterize a crystalline solid defined by repetition of a unit cell or elementary lattice on a molecular scale. It follows the same physical principle as that governing the small angle X-ray diffraction technique. Thus, the wide angle XRD technique is used to analyse the materials used in accordance with the invention because it is particularly suited to the structural characterization of metal oxide particles which might be crystalline present in each of the elementary spherical particles constituting the material used in accordance with the invention.

Nitrogen volumetric analysis, corresponding to the physical adsorption of molecules of nitrogen in the pores of the material via a gradual increase in the pressure at constant temperature, provides information on the textural characteristics (pore diameter, pore volume, specific surface area) particular to the material of the invention. In particular, it provides access to the specific surface area and to the mesopore distribution used in the material. The term “specific surface area” means the BET specific surface area (S_{BET} in m²/g) determined by nitrogen adsorption in accordance with ASTM standard D 3663-78 derived from the BRUNAUER-EMMETT-TELLER method described in the periodical “The Journal of the American Society”, 1938, 60, 309. The pore distribution which is representative of a population of mesopores centred on a range of 2 to 50 nm (IUPAC classification) is determined from the Barrett-Joyner-Halenda model (BJH). The nitrogen adsorption-desorption isotherm in accordance with the BJH model which is obtained is described in the periodical “The Journal of the American Society”, 1951, 73, 373, written by E. P. Barrett, L. G. Joyner and P. P. Halenda. In the disclosure below, the diameter 0 of the mesopores of the mesostructured matrix corresponds to the maximum diameter read on the pore size distribution curve obtained from the adsorption branch of the nitrogen isotherm. In addition, the shape of the nitrogen adsorption isotherm and of the hysteresis loop can provide information on the nature of the mesopores in the material used in accordance with the invention.

Concerning the mesostructured matrix, the difference between the value for the pore diameter φ and the lattice parameter, a, defined by small angle XRD as described above can be used to provide a quantity e, where e=a−φ, which is characteristic of the thickness of the amorphous walls of the mesostructured matrix included in each of the spherical particles of the material used in accordance with the invention. Said lattice parameter, a, is linked to the correlation distance d between pores by a geometric factor which is characteristic of the geometry of the phase. As an example, in the case of a vermicular structure, e=d−φ.

Transmission electron microscopy (TEM) is also a technique which is used to characterize the structure of these materials. It can be used to form an image of the solid being studied, the contrasts observed being characteristics of the structural organization, the texture or indeed the morphology of the elementary particles observed; the maximum resolution of the technique is 1 nm. This technique can also be used to visualize the metal oxide particles trapped in said mesostructured matrix, provided that they have a size which is larger than the detection limit, i.e. a size of more than 1 nm. In the case in which said metal oxide particles trapped in said mesostructured matrix are less than 1 nm in size, coupling with an elemental analysis such as EDX, or energy dispersive X ray spectroscopy, can be used to detect the presence of a metallic element. In the disclosure below, TEM photographs were taken from microtome sections of a sample in order to observe a section of an elementary spherical particle of the material used in accordance with the invention. Image analysis can also be used to provide access to the parameters d, φ and e which are characteristic of the mesostructured matrix defined above.

The morphology and the size distribution of the elementary particles were established by analysis of the photos obtained by scanning electron microscopy (SEM).

The metal oxide particles as described below in the present disclosure were in particular characterized by Raman spectroscopy. The Raman spectra were obtained with a dispersive type Raman spectrometer equipped with a laser with an excitation wavelength of 532 nm. The laser beam was focussed on the sample using a microscope provided with a x 50 long working distance objective. The power of the laser at the sample was of the order of 1 mW. The Raman signal emitted by the sample was collected by the same objective and dispersed using a 1800 line/mm grating then collected by a CCD (Charge Coupled Device or a charge transfer device) detector. The spectral resolution obtained was of the order of 2 cm⁻¹. The spectral zone recorded was between 300 and 1500 cm⁻¹. The acquisition period was fixed at 120 s for each Raman spectrum recorded.

Nuclear magnetic resonance (NMR) was also advantageously used to characterize the material used in accordance with the invention. In particular, ³¹P, ²⁷Al and ²⁹Si NMR analyses recorded on 300 or 400 MHz spectrometers can be mentioned. Nuclear magnetic resonance (NMR) spectroscopy, in particular ⁹⁵Mo and ¹⁸³W NMR, was also advantageously used to characterize the metal oxide particles described below in the present disclosure.

### Metathesis Reaction, and Associated Process

The present invention concerns a process for the metathesis of olefins, comprising bringing said olefins into contact with said catalyst which has been activated by heating to a temperature in the range 100° C. to 1000° C. in an atmosphere of a non-reducing gas, in particular in particular for the following reactions, the list of which is non-limiting: ring closing by metathesis, acyclic diene polymerization by metathesis, ring opening polymerization using metathesis, acyclic olefin metathesis, cross metathesis of cyclic and acyclic olefins and the metathesis of functionalized olefins.

The olefins used in the metathesis process of the invention are advantageously selected from monoolefins containing 2 to 30 carbon atoms, preferably 2 to 25 carbon atoms and still more preferably 2 to 18 carbon atoms such as, for example, ethylene, propylene, butenes, pentenes, cycloolefins containing 3 to 20 carbon atoms such as cyclopentene, cyclooctene, norbornene, for example, polyolefins containing 4 to 30 carbon atoms such as 1,4-hexadiene, 1,7-octadiene, for example, and cyclopolyolefins containing 5 to 30 carbon atoms such as 1,5-cyclooctadiene, norbornadiene, dicyclopentadiene, said olefins being used alone or as a mixture, said monoolefins or polyolefins, which may be linear or cyclic, advantageously being functionalized with functional groups such as halogens, or ester, hydroxyl, ketone, aldehyde, ether, carboxyl, amine, amide, anhydride, nitrile, phosphine or phosphite groups, for example.

Preferably, the olefins used in the metathesis process of the invention are advantageously selected from ethylene and butenes, used alone or as a mixture, functionalized or otherwise, and preferably a mixture of ethylene and butenes, which may or may not be functionalized.

The olefin metathesis process of the invention consists of bringing olefins, advantageously in the liquid or gaseous state, into contact with a solid catalyst as described in the invention. The process consists of:

a) a catalyst activation step;

b) the metathesis reaction proper.

In accordance with the invention, said catalyst has been activated by heating to a temperature in the range 100° C. to 1000° C., preferably in the range 300° C. to 600° C., in an atmosphere of a non-reducing gas advantageously selected from oxygen, synthetic air, nitrogen, argon, helium and oxygen diluted with nitrogen, preferably in nitrogen, preferably under static or dynamic conditions, and preferably in a light stream of gas. The moisture content of the gas stream is preferably kept below 200 ppm (parts per million). The duration of this activation treatment is preferably in the range ten minutes to ten hours or more. After this, the active catalyst obtained is cooled in an atmosphere which is preferably anhydrous. Advantageously, a nitrogen purge may be carried out before contact with the hydrocarbon feed. Activation of said catalyst may advantageously be carried out directly inside the reactor or outside the reactor before transferring the catalyst to the reactor.

The metathesis process of the invention is advantageously carried out by bringing the olefins into contact with said catalyst in the liquid phase or in the gaseous phase independently of the structure and the molecular mass of the olefins.

The metathesis process of the invention may advantageously be carried out in a stirred reactor or by passing the olefin reagent or reagents through a fixed, moving or fluidized bed of said catalyst.

The metathesis process of the invention is preferably operated at a temperature in the range −20° C. to 200° C., preferably in the range 20° C. to 140° C., under pressure conditions which vary as a function of whether the reaction is to be carried out in the gas phase or in the liquid phase.

In a liquid phase operation, the pressure must be sufficient for the majority (more than 50%) of the reagents and any solvent to be maintained in the liquid phase (or in the condensed phase). The catalyst can then advantageously be used either in pure olefin (or olefins) or in the presence of a solvent constituted by an aliphatic, cycloaliphatic or aromatic hydrocarbon, a halogenated hydrocarbon or a nitro derivative. Preferably, a hydrocarbon or a halogenated hydrocarbon is used.

The following examples illustrate the present invention without in any way limiting its scope.

### Example 1 (in Accordance with the Invention)

### Preparation of a Material a Used in Accordance with the Invention Containing 10% by Weight with Respect to the Final Material of Metal Oxide Particles of Molybdenum MoO₃ Trapped in a Mesostructured Matrix Oxide Based on Silicon and Aluminium with a Si/al Molar Ratio of 12 Using Keggin Type HPA Precursors with Formula H₃PMo₁₂O₄₀ (Commercial) (Template or Surfactant P123)

An aqueous solution containing 0.41 g of H₃PMo₁₂O₄₀ and 15 g of permuted water was prepared, with stirring, at ambient temperature. 1.09 g of aluminium trichloride was mixed with 19.3 g of permuted water and 7.00 mg of HCl. After stirring for 5 minutes at ambient temperature, 11.2 g of TEOS was added. The solution was allowed to hydrolyse for 16 h, with stirring at ambient temperature (solution of matrix precursors). Another solution was prepared by mixing 3.56 g of P123 in 35.9 g of permuted water, 13.1 mg of HCl and 8.48 g of ethanol. The aquo-organic solution of P123 was then added to the solution of matrix precursors. After homogenizing for 5 min, the solution containing the H₃PMo₁₂O₄₀ was added dropwise to the solution of matrix precursors. The mixture was stirred for 30 min then sent to the atomisation chamber of the aerosol generator and the solution was sprayed in the form of fine droplets under the action of a vector gas (dry air) introduced under pressure (P=1.5 bars). The droplets were dried using the protocol described in the above disclosure of the invention. The temperature of the drying furnace was fixed at 350° C. The ³¹P NMR spectrum of the material at this stage revealed the presence of the Keggin HPA H₃PMo₁₂O₄₀ with a single characteristic peak for this heteropolyanion at −3.8 ppm. The harvested powder was then calcined in air for 12 h at T=550° C. The solid was characterized by small angle XRD, by nitrogen volumetric analysis, by TEM and by XRF. The TEM analysis showed that the final material had an organized mesoporosity characterized by a vermicular structure. In addition, this analysis could not be used to detect the presence of any metal oxide particles, meaning that the particles were less than 1 nm in size. An EDX analysis coupled with the TEM clearly confirmed the presence of metal. The nitrogen volumetric analysis resulted in a specific surface area for the final material of S_{BET}=194 m²/g and to a mesopore diameter of 7.1 nm. Small angle XRD analysis led to observation of a correlation peak at the angle 2θ=0.80. The Bragg relation, 2 d*sin (0.40)=1.5406, was used to calculate the correlation distance, d, between the pores of the mesostructured matrix, i.e. d=10.9 nm. The thickness of the walls of the mesostructured material, defined by e=d−φ, was thus e=3.8 nm. The molar ratio Si/Al obtained by XRF was 12. A SEM image of the spherical elementary particles of the final material obtained indicated that these particles had a size characterized by a diameter of 50 nm to 30 μm, the size distribution of these particles being centred about 15 p.m. The Raman spectrum of the final material revealed the presence of polymolybdate species, in interaction with the matrix, with characteristic bands for these species at 950 cm⁻¹.

### Example 2 (in Accordance with the Invention)

### Preparation of a Material B Used in Accordance with the Invention Containing 6% by Weight with Respect to the Final Material of Metal Oxide Particles of Molybdenum MoO₃ Trapped in a Mesostructured Matrix Oxide Based on Silicon and Aluminium with a Si/al Molar Ratio of 12 Using the Monometallic Precursor MoCl₅ (Template F127)

An aqueous solution containing 0.29 g of MoCl₅ and 15.0 g of permuted water was prepared, with stirring, at ambient temperature. 0.69 g of aluminium trichloride was mixed with 12.1 g of permuted water and 4.40 mg of HCl. After stirring for 5 minutes at ambient temperature, 7.16 g of TEOS was added. The solution was allowed to hydrolyse for 16 h, with stirring at ambient temperature (solution of matrix precursors). Another solution was prepared by mixing 2.41 g of F127 in 22.4 g of permuted water, 8.20 mg of HCl and 5.29 g of ethanol. The aquo-organic F127 solution was then added to the solution of matrix precursors. After homogenizing for 5 min, the solution containing the MoCl₅ was added dropwise to the solution of matrix precursors. The mixture was stirred for 30 min then sent to the atomisation chamber of the aerosol generator and the solution was sprayed in the form of fine droplets under the action of a vector gas (dry air) introduced under pressure (P=1.5 bars). The droplets were dried using the protocol described in the above disclosure of the invention. The temperature of the drying furnace was fixed at 350° C. The harvested powder was then calcined in air for 12 h at T=550° C. The solid was characterized by small angle XRD, by nitrogen volumetric analysis, by TEM and by XRF. The TEM analysis showed that the final material had an organized mesoporosity characterized by a vermicular structure. In addition, this analysis could not be used to detect the presence of any metal oxide particles, meaning that the particles were less than 1 nm in size. An EDX analysis coupled with the TEM clearly confirmed the presence of metal. The nitrogen volumetric analysis resulted in a specific surface area for the final material of S_{BET}=160 m²/g and a mesopore diameter of 9.6 nm. Small angle XRD analysis led to observation of a correlation peak at the angle 2θ=0.70. The Bragg relation 2 d*sin (0.35)=1.5406 was used to calculate the correlation distance, d, between the pores of the mesostructured matrix, i.e. d=12.6 nm. The thickness of the walls of the mesostructured material, defined by e=d−φ, was thus e=3 nm. The molar ratio Si/Al obtained by XRF was 12. A SEM image of the spherical elementary particles of the final material obtained indicated that these particles had a size characterized by a diameter of 50 nm to 30 μm, the size distribution of these particles being centred about 15 μm. The Raman spectrum of the final material revealed the presence of polymolybdate species, in interaction with the matrix, with characteristic bands for these species at 950 cm⁻¹.

### Example 3 (in Accordance with the Invention)

### Preparation of a Material C Used in Accordance with the Invention Containing 10% by Weight with Respect to the Final Material of Metal Oxide Particles of Molybdenum MoO₃ Trapped in a Mesostructured Matrix Oxide Based on Silicon and Aluminium with a Si/al Molar Ratio of 12 Using Metallic Keggin Type HPA Precursors with Formula H₃PMo₁₂O₄₀ (Template Brij58)

An aqueous solution containing 0.41 g of H₃PMo₁₂O₄₀ (commercial) and 15.0 g of permuted water was prepared, with stirring, at ambient temperature. 1.09 g of aluminium trichloride was mixed with 19.3 g of permuted water and 7.00 mg of HCl. After stirring for 5 minutes at ambient temperature, 11.2 g of TEOS was added. The solution was allowed to hydrolyse for 16 h, with stirring at ambient temperature (solution of matrix precursors). Another solution was prepared by mixing 3.56 g of Brij58 in 35.9 g of permuted water, 13.1 mg of HCl and 8.48 g of ethanol. The aquo-organic solution of Brij58 was then added to the solution of matrix precursors. After homogenizing for 5 min, the solution containing the H₃PMo₁₂O₄₀ was added dropwise to the solution of matrix precursors. The mixture was stirred for 30 min then sent to the atomisation chamber of the aerosol generator and the solution was sprayed in the form of fine droplets under the action of a vector gas (dry air) introduced under pressure (P=1.5 bars). The droplets were dried using the protocol described in the above disclosure of the invention. The temperature of the drying furnace was fixed at 350° C. The ³¹P NMR spectrum of the material at this stage revealed the presence of the Keggin HPA H₃PMo₁₂O₄₀ with a single characteristic peak for this heteropolyanion at −3.8 ppm. The harvested powder was then calcined in air for 12 h at T=550° C. The solid was characterized by small angle XRD, by nitrogen volumetric analysis, by TEM and by XRF. The TEM analysis showed that the final material had an organized mesoporosity characterized by a vermicular structure with a pore size of 2 nm. In addition, this analysis could not be used to detect the presence of any metal oxide particles, meaning that the particles were less than 1 nm in size. An EDX analysis coupled with the TEM clearly confirmed the presence of metal. The nitrogen volumetric analysis resulted in a specific surface area for the final material of S_{BET}=480 m²/g and a pore volume of 0.28 mL/g. Small angle XRD analysis produced a correlation distance d between the pores of the mesostructured matrix, i.e. d=4.6 nm. The thickness of the walls of the mesostructured material, defined by e=d−φ, was thus e=2.6 nm. The molar ratio Si/Al obtained by XRF was 12. A SEM image of the spherical elementary particles of the final material obtained indicated that these particles had a size characterized by a diameter of 50 nm to 30 μm, the size distribution of these particles being centred about 15 μm. The Raman spectrum of the final material revealed the presence of polymolybdate species, in interaction with the matrix, with characteristic bands for these species at 950 cm⁻¹.

### Example 4 (in Accordance with the Invention)

### Preparation of a Material D Used in Accordance with the Invention, Containing 10% by Weight with Respect to the Final Material of Metal Oxide Particles of Molybdenum MoO₃ Trapped in a Mesostructured Matrix Oxide Based on Silicon and Aluminium with a Si/al Molar Ratio of 12 Using Preformed MoO₃ Particles (Template P123)

A suspension composed of 0.44 g of Mo(CO)₆, 16.8 mL of phenoxybenzene and 0.84 mL of oleic acid was slowly heated from ambient temperature to 310° C. in an inert atmosphere for 25 min. Gaseous oxygen (flow rate: 200 mL/min) was bubbled through the solution at 310° C. for 30 min. After cooling the colloidal solution, 20 g of ethanol was added to the solution (protocol adapted from Park et al. Chem. Mater. 2007, 19, 2706). 0.68 g of aluminium trichloride was mixed with 12.1 g of permuted water and 4.40 mg of HCl. After stirring for 5 minutes at ambient temperature, 7.02 g of TEOS was added. The solution was allowed to hydrolyse for 16 h, with stirring at ambient temperature (solution of matrix precursors). Another solution was prepared by mixing 2.22 g of P123 in 22.4 g of permuted water, 8.20 mg of HCl and 15.32 g of ethanol. The aquo-organic solution of P123 was then added to the solution of matrix precursors. After homogenizing for 5 min, the colloidal solution of MoO₃ nanoparticles was added dropwise. The mixture was stirred for 30 min then sent to the atomisation chamber of the aerosol generator and the solution was sprayed in the form of fine droplets under the action of a vector gas (dry air) introduced under pressure (P=1.5 bars). The droplets were dried using the protocol described in the above disclosure of the invention. The temperature of the drying furnace was fixed at 350° C. The harvested powder was then calcined in air for 12 h at T=550° C. The solid was characterized by small angle XRD, by nitrogen volumetric analysis, by TEM and by XRF. The TEM analysis showed that the final material had an organized mesoporosity characterized by a vermicular structure. In addition, this analysis was used to detect the presence of metal oxide particles of MoO₃ with a mean size of 2.5 nm. The nitrogen volumetric analysis resulted in a specific surface area for the final material of S_{BET}=155 m²/g and a mesopore diameter of 6.8 nm. Small angle XRD analysis led to observation of a correlation peak at the angle 28=0.80. The Bragg relation, 2 d*sin (0.40)=1.5406, was used to calculate the correlation distance, d, between the pores of the mesostructured matrix, i.e. d=10.9 nm. The thickness of the walls of the mesostructured material, defined by e=d−φ, was thus e=4.1 nm. The molar ratio Si/Al obtained by XRF was 12. A SEM image of the spherical elementary particles of the final material obtained indicated that these particles had a size characterized by a diameter of 50 nm to 30 μm, the size distribution of these particles being centred about 15 μm.

### Example 5 (in Accordance with the Invention)

### Preparation of a Material E Used in Accordance with the Invention Containing 10% by Weight with Respect to the Final Material of Metal Oxide Particles of Molybdenum MoO₃ Trapped in a Mesostructured Matrix Oxide Based on Silicon and Aluminium with a Si/al Molar Ratio of 12 Using MoCl₅ Type Metallic Precursors (Template Brij58)

An aqueous solution containing 0.29 g of MoCl₅ and 15.0 g of permuted water was prepared, with stirring, at ambient temperature. 1.09 g of aluminium trichloride was mixed with 19.3 g of permuted water and 7.00 mg of HCl. After stirring for 5 minutes at ambient temperature, 11.2 g of TEOS was added. The solution was allowed to hydrolyse for 16 h, with stirring at ambient temperature (solution of matrix precursors). Another solution was prepared by mixing 3.56 g of Brij58 in 35.9 g of permuted water, 13.1 mg of HCl and 8.48 g of ethanol. The aquo-organic solution of P123 was then added to the solution of matrix precursors. After homogenizing for 5 min, the solution containing the H₃PMo₁₂O₄₀ was added dropwise to the solution of matrix precursors. The mixture was stirred for 30 min then sent to the atomisation chamber of the aerosol generator and the solution was sprayed in the form of fine droplets under the action of a vector gas (dry air) introduced under pressure (P=1.5 bars). The droplets were dried using the protocol described in the above disclosure of the invention. The temperature of the drying furnace was fixed at 450° C. The harvested powder was then calcined in air for 12 h at T=550° C. The solid was characterized by small angle XRD, by nitrogen volumetric analysis, by TEM and by XRF. The TEM analysis showed that the final material had an organized mesoporosity characterized by a vermicular structure with a pore size of 2 nm. In addition, this analysis could not be used to detect the presence of any metal oxide particles, meaning that the particles were less than 1 nm in size. X ray diffraction analysis clearly confirmed the presence of metal. The nitrogen volumetric analysis resulted in a specific surface area for the final material of S_{BET}=480 m²/g and a pore volume of 0.28 mL/g. Small angle XRD analysis produced a correlation distance d between the pores of the mesostructured matrix, i.e. d=4.6 nm. The thickness of the walls of the mesostructured material, defined by e=d−φ, was thus e=2.6 nm. The molar ratio Si/Al obtained by XRF was 12. A SEM image of the spherical elementary particles of the final material obtained indicated that these particles had a size characterized by a diameter of 50 nm to 30 μm, the size distribution of these particles being centred about 15 μm. The Raman spectrum of the final material revealed the presence of polymolybdate species, in interaction with the matrix, with characteristic bands for these species at 950 cm¹.

### Example 6 (not in Accordance with the Invention)

### Preparation of a Material F Containing 6% by Weight with Respect to the Final Material of Molybdenum Oxide, MoO₃, in a Mesoporous Oxide Matrix Based on Silicon and Aluminium with a Si/al Molar Ratio of 10 by Wet Impregnation of MoO₃ Particle Precursors onto a Preformed Aluminosilicate Solid

The operating protocol was adapted from Debecker et al. Catal. Today, 2011, 169, 1, 60. The silica-alumina support (Aldrich Grade 135, S_{BET}=490 m²/g, pore volume=0.71 mL/g, 13% by weight of alumina) was calcined at 500° C. before impregnation and then kept at 110° C. 5 g of support was suspended in an aqueous solution of ammonium heptamolybdate (200 mL) with a concentration adapted to the envisaged final charge. The suspension was stirred for 2 h. The water was evaporated off under vacuum using a rotary evaporator. The recovered solid was dried overnight at 110° C. then calcined in air at 400° C. The catalyst was characterized by nitrogen volumetric analysis, wide angle XRD, TEM and NMR. Following impregnation, the specific surface area and the pore volume dropped to 400 m²/g and 0.56 mL/g respectively, but the mean pore diameter was unaffected. A portion of the deposited molybdenum oxide was located outside the support particles. Wide angle X ray diffraction demonstrated the presence of crystals of Mo oxide. Their presence outside the support particles was confirmed by TEM observation. ²⁷Al NMR revealed the presence of Al₂(MoO₄)₃ in abundance.

### Example 7 (not in Accordance with the Invention)

### Preparation of a Material G Containing 10% by Weight with Respect to the Final Material of Molybdenum Oxide, MoO₃, in a Mesoporous Oxide Matrix Based on Silicon and Aluminium with a Si/al Molar Ratio of 10 by Wet Impregnation of MoO₃ Particle Precursors onto a Preformed Aluminosilicate Solid

The operating protocol was adapted from Debecker et al. Catal. Today, 2011, 169, 1, 60. The silica-alumina support (Aldrich Grade 135, S_{BET}=490 m²/g, pore volume=0.71 mL/g, 13% by weight of alumina) was calcined at 500° C. before impregnation and then kept at 110° C. 5 g of support was suspended in an aqueous solution of ammonium heptamolybdate (200 mL) with a concentration adapted to the envisaged final charge. The suspension was stirred for 2 h. The water was evaporated off under vacuum using a rotary evaporator. The recovered solid was dried overnight at 110° C. then calcined in air at 400° C. The catalyst was characterized by nitrogen volumetric analysis, wide angle XRD, TEM and MMR. Following impregnation, the specific surface area and the pore volume dropped to 400 m²/g and 0.56 mL/g respectively, but the mean pore diameter was unaffected. A portion of the deposited molybdenum oxide was located outside the support particles. Wide angle X ray diffraction demonstrated the presence of crystals of Mo oxide. Their presence outside the support particles was confirmed by TEM observation. ²⁷Al NMR revealed the presence of Al₂(MoO₄)₃ in abundance.

### Example 8

### Catalytic Butene and Ethylene Metathesis Reaction Using Catalysts a to G

Materials A to G were obtained in the powder form and were shaped by pelletization and crushing the synthesized powder so that it could be used as catalysts A to G in the olefin metathesis process of the invention. Catalysts A to G employed were pressed, ground and screened into the 200-315 μm granulometric fraction and introduced (200 mg) into a 5 mm internal diameter quartz reactor. They were activated under nitrogen (20 mL/min) at 550° C. for 2 hours. Still under a stream of nitrogen, they were cooled to the reaction temperature, i.e. 40° C. An equimolar stream (8 mL/min) of ethylene and trans-2-butene was then introduced. The streams of nitrogen and olefins were purified using Molsieve 3A (Roth) and Oxysorb-glass (Linde) filters. During the reaction, carried out at atmospheric pressure, the composition of the effluent gas was measured in-line using an Agilent 6890 GC gas chromatograph. Analysis of the products took approximately 7 min for each injection. Product separation was carried out in a HP-AL/M column (30 m long, 0.53 mm internal diameter, 0.15 film thickness) with a temperature ramp-up from 90° C. to 140° C. and with a flame ionization detector.

The ethylene and the 2-butenes were converted into propene via the metathesis reaction with a selectivity of the order of 99%. Only traces of secondary metathesis products (1-butene, pentenes, hexenes) or isomerization products (isobutene) were detected. The specific activity is defined as the number of moles of propene produced per g of catalyst per hour. The number of moles of propene produced per g of catalyst per hour was measured conventionally by gas chromatography. The activity was measured over one hour. The results are given for the reaction time of 14 minutes (Table 1).

TABLE 1
Specific activity of catalysts A-G in the metathesis of
ethylene and butene

| Catalyst | Specific activity (mmole.g⁻¹.h⁻¹) |
|---|---|
| A | 25 |
| B | 23 |
| C | 32 |
| D | 15 |
| E | 26 |
| F not in accordance | 11 |
| G not in accordance | 14 |

1. A process for the metathesis of olefins, comprising bringing said olefins into contact with a catalyst which has been activated by heating to a temperature in the range 100° C. to 1000° C. in an atmosphere of non-reducing gas, said catalyst comprising at least one inorganic material constituted by at least two elementary spherical particles, each of said elementary spherical particles comprising metal oxide particles with a size of at most 300 nm and containing at least one metal selected from tungsten, molybdenum, rhenium, cobalt, tin, ruthenium, iron and titanium, used alone or as a mixture, said metal oxide particles being present within a mesostructured matrix based on an oxide of at least one element Y selected from silicon, aluminium, titanium, tungsten, zirconium, gallium, germanium, tin, antimony, lead, vanadium, iron, manganese, hafnium, niobium, tantalum, yttrium, cerium, gadolinium, europium and neodymium and a mixture of at least two of these elements, said mesostructured matrix having a pore size in the range 1.5 to 50 nm and having amorphous walls with a thickness in the range 1 to 30 nm and said elementary spherical particles having a maximum diameter of 200 μm.

2. A process according to claim 1, in which said process operates at a temperature in the range −20° C. to 200° C.

3. A process according to claim 1, in which the size of said metal oxide particles is at most 50 nm.

4. A process according to claim 3, in which the size of said metal oxide particles is at most 3 nm.

5. A process according to claim 1, in which said metal oxide particles contain at least one metal selected from tungsten, molybdenum and rhenium, used alone or as a mixture.

6. A process according to claim 5, in which said metal oxide particles contain molybdenum alone.

7. A process according to claim 1, in which said metal oxide particles are trapped in said mesostructured matrix.

8. A process according to claim 1, in which said metal oxide particles are obtained from precursors of said metal oxide particles selected from polyoxometallates with formula (XₓMₘO_{y}Hₕ)^{q-} (I) where H is the hydrogen atom, O is the oxygen atom, X is an element selected from rhenium, phosphorous, silicon, boron, nickel, tin, ruthenium, iron, titanium and cobalt and M is one or more elements selected from molybdenum, tungsten, rhenium, cobalt, tin, ruthenium, iron and titanium, x being equal to 0, 1, 2, or 4, m being equal to 5, 6, 7, 8, 9, 10, 11, 12 and 18, y being in the range 17 to 72, h being in the range 0 to 12 and q being in the range 1 to 20, y, h and q being whole numbers

9. A process according to claim 8, in which said metallic precursors of said metal oxide particles are heteropolyanions with formula XM₁₂O₄₀Hₕ^{q-} (x=1, m=12, y=40, h=0 to 12, q=3 to 12) and/or the formula XM₁₁O₃₉Hₕ^{q-} (x=1, m=11, y=39, h=0 to 12, q=3 to 12).

10. A process according to claim 8, in which said metallic precursors of said metal oxide particles are heteropolyanions with formula XM₆O₂₄Hₕ^{q-} (with x=1, m=6, y=24, q=3 to 12 and h=0 to 12) and/or the formula X₂M₁₀O₃₈Hₕ^{q-} (with x=2, m=10, y=38, q=3 to 12 and h=0 to 12).

11. A process according to claim 8, in which said metal precursors of said metal oxide particles are heteropolyanions with formula P₂Mo₅O₂₃Hₕ^{(6-h)-}, in which h=0, 1 or 2.

12. A process according to claim 1, in which said metal oxide particles are obtained from precursors of said metal oxide particles selected from monometallic precursors.

13. A process according to claim 12, in which said monometallic precursors are selected from the following species: (NH₄)₂MO₄ (M=Mo, W), Na₂MO₄ (M=Mo, W), H₂MoO₄, (NH₄)₂MS₄ (M=Mo, W), MoO₂Cl₂, MoCl₄, MoCl₅, Na₂MoO₄, (NH₄)Mo₂O₇, Mo(NO₂)Cl₂, W(OEt)₅, W(Et)₆, WCl₆, WCl₄, WCl₂, WPhCl₃, NH₄ReO₄, Re₂(CO)₁₀, HReO₄, ReCl₅, Bu₄Sn, SnCl₄, Sn(C₂H₅)₄, RuCl₃, Ru₃(CO)₁₂, Ru(NO)(NO₃)₃, 2CoCO₃.3Co(OH)₂—H₂O, Co(NO₃)₂.6H₂O, CoCl₂, Fe(NO₃)₃, FeSO₄, FeCl₂.4H₂O, FeCl₂, FeCl₃, Fe(CO)₅, Fe₂O₃, Fe₃O₄, TiCl₄, Ti(OCOCCl₃)₃, TiF₄, Ti(OⁱPr)₄, and TiCl(OᵢPr)₃.

14. A process according to claim 1, in which said mesostructured matrix is preferably constituted by aluminium oxide, silicon oxide or a mixture of silicon oxide and aluminium oxide.

15. A process according to claim 1, in which the olefins used in said process are selected from ethylene and butenes, used alone or as a mixture, which may or may not be functionalized.
